(19) United States
(12) Patent Application Publication
(10) Pub. No.: US 2007/0270875 A1
(43) Pub. Date: Nov. 22, 2007

(54) Medical Instrument For Spreading Vertebral Bodies Apart

(76) Inventors: Uwe Bacher, Tuttlingen (DE); Martin Oberlaender, Engen (DE)

Correspondence Address: ST. ONGE STEWARD JOHNSTON & REENS, LLC, 986 BEDFORD STREET, STAMFORD, CT, 06905-5619, US

(21) Appl. No.: 11/735,172

(22) Filed: Apr. 13, 2007

(30) Foreign Application Priority Data
Apr. 13, 2006 (DE) 10 2006 018 248.0

(51) Int. Cl. A61B 17/58

(52) U.S. Cl. 606/90

(57) ABSTRACT

A medical instrument serves for spreading vertebral bodies apart and for operations in an area of a spinal column. A sleeve is provided and several spreadable elements protrude from a distal end of said sleeves. Said several spreadable elements are folded together to form a conical body, said conical body serves for inserting between adjacent vertebral bodies of a spinal column. A spreader is provided which can be driven into said conical body thereby spreading said several spreading elements, said spreading elements pushing away said two adjacent vertebral bodies.

### BACKGROUND OF THE INVENTION

The invention relates to a medical instrument for spreading vertebral bodies apart and for operations in the area of a spinal column.

A device of this kind in the form of an instrument set for insertion of a surgical implant is known from DE 697 29 140 T2.

Such medical instruments are used for spreading two adjacent vertebrae apart during an examination or a surgical intervention in the area of a spinal column, for example for inserting an implant.

To do this, two elements protruding from the distal end of the cannula are inserted between two adjacent vertebrae in such a way that the two adjacent vertebrae are pushed away from one another. The adjacent vertebrae are kept spaced apart by the two elements throughout the operation. Various medical instruments and an implant can be guided through the working channel passing through the cannula and can be brought to the specified site between the adjacent vertebrae.

The two distally protruding elements are designed as strip-shaped extensions of the cannula wall. At the distal end, they are pointed in order to make it easier to insert the cannula between the vertebral bodies. The height or width of the strips defines the distance to which the vertebrae can be spread.

From EP 0 767 636 B1 a vertebral implant is known which can be inserted into the inter-vertebral joint space. Two superposed branches can be parted from one another via a screw. A posterior height of the implant remains constant and an anterior height can be varied for spreading two adjacent vertebrae.

It is an object of the present invention to develop a medical instrument in such a way as to ensure simple insertion of the instrument between two adjacent vertebrae and a sufficient operating area during the examination or the surgical intervention.

### SUMMARY OF THE INVENTION

This object is achieved by a medical instrument for spreading vertebral bodies apart and for operations in an area of a spinal column which comprises a sleeve, several spreadable elements protruding from a distal end of said sleeve, said several spreadable elements are folded together to form a conical body, said conical body is inserted between adjacent vertebral bodies of a spinal column, and a spreader which can be driven into said conical body thereby spreading that several spreading elements, said spreading elements pushing away said two adjacent vertebral bodies.

These measures have the considerable advantage, among other things, that the conical body can first be driven in between the vertebrae, and it is only thereafter that the conical body and thus the vertebrae are spread by insertion of the spreader. Due to the cone geometry, the surgeon can place a tip of the cone at the spinal column without considering the rotational position of the cone.

The surgeon can fully concentrate on the exact location between the two adjacent vertebrae followed by pushing the conical body between the vertebrae.

The spreader is now inserted into the conical body for spreading the several elements folded to said conical body. This gives the operator a good feeling for the extent of the spreading. There is not only a spreading in axially opposite directions but also a spreading in lateral direction. This opens a large area for surgical interventions in the spinal column.

The sleeve can be gripped by a human hand, with the palm and fingers closing firmly around the sleeve. In this way, the instrument can be held very securely and safely. This permits ergonomic handling of the medical instrument when inserting the conical body between two adjacent vertebrae.

In another embodiment of the invention, the spreader is designed as a cannula that is received displaceably in the sleeve.

This measure has the advantage that the cannula inserted into the sleeve constitutes a support and a reinforcement for the spreadable elements when they are spread open. The spread-open vertebrae exert a considerable pressing force on the spread elements. Additionally, the inner space of the cannula allows to inserted instruments therethrough.

In another embodiment of the invention, a penetrating element is provided that can be guided through the cannula and through the conical body.

This measure has the advantage that the penetrating element simplifies the targeting of the site of application of the instrument and the insertion of the conical body between two adjacent vertebrae.

In another embodiment of the invention, the conical body is designed as a truncated cone.

In this embodiment, an opening is created at the distal end through which an exact positioning between the vertebrae can be obtained, for example by means of a target wire being guided through it.

In another embodiment of the invention, the penetrating element is designed as a rod.

This measure has the advantage that the rod can be pushed by one hand through the instrument and out past the distal end thereof.

In another embodiment of the invention, the penetrating element tapers in the direction of its distal end.

This measure has the advantage that the tapered shape of the distal end of the rod facilitates the insertion of the penetrating element between two adjacent vertebrae.

In another embodiment of the invention, the tapering distal end of the rod is designed as a point.

This measure has the advantage that the distal end designed as a point can be applied at a precise site and driven into place.

In another embodiment of the invention, the spreadable elements are designed as blades.

Blades can easily be folded together to form the conical body by being bent radially inward into a conical cage.

In another embodiment of the invention, the blades are formed by slits in the sleeve.

This measure has the advantage that the spreadable elements can be produced by simple machining of the sleeve.

In another embodiment of the invention, each blade is connected to the sleeve via a hinge.

This measure has the advantage that the blades are pivoted about the hinge in a defined manner, specifically when pivoting inward to form the conical body and also when spread open by the cannula. The hinge is designed in particular as a film hinge.

In another embodiment of the invention, the blades are bent radially inward.

This measure has the advantage that the elements automatically fold together to the conical body after the spreader is withdrawn.

In another embodiment of the invention, the blades have an outer barb at their distal end.

This measure has the advantage that the barbs serve to create defined sites of force introduction during spreading of the blades.

In another embodiment of the invention, when the blades are spread open, the outer faces of the barbs form a ring whose external diameter is greater than the external diameter of the sleeve.

This measure has the advantage that the maximum extent of spreading can be greater than the external diameter of the sleeve. Variable extents of spreading can be achieved through the choice of the shape and height of the barbs. By means of the ring-shaped body that is obtained, the spreading force can be led off uniformly to the vertebrae.

It will be appreciated that the aforemeintioned features and the features still to be explained below can be used not only in the respectively cited combination but also in other combinations or singly, without departing from the scope of the present invention.

### BRIEF DESCRIPTION OF THE DRAWINGS

The invention is described and explained in more detail below on the basis of a selected illustrative embodiment and with reference to the drawings, in which:

FIG. 1 shows a perspective side view of a medical instrument according to the invention, in which the spreadable elements are folded together to form a distally tapering body,

FIG. 2 shows a view comparable to the view in FIG. 1, with a penetrating element having been pushed in,

FIG. 3 shows a view comparable to FIG. 2, after the spreadable elements have been spread open,

FIG. 4 shows a cross section along the longitudinal axis in FIG. 2,

FIG. 5 shows a cross section along the longitudinal axis in FIG. 3,

FIG. 6 shows a medical instrument before it is inserted between two adjacent vertebrae,

FIG. 7 shows a medical instrument that has been inserted between two adjacent vertebrae, before the spreadable elements are spread open,

FIG. 8 shows a view comparable to the view in FIG. 7, after the spreadable elements have been spread open, and

FIG. 9 shows a view comparable to the view in FIG. 7, after the penetrating element has been withdrawn.

### DETAILED DESCRIPTION OF PREFERRED EMBODIMENTS

In the figures, a medical instrument for spreading vertebral bodies apart and for operations in the area of a spinal column is designated overall by reference number 10.

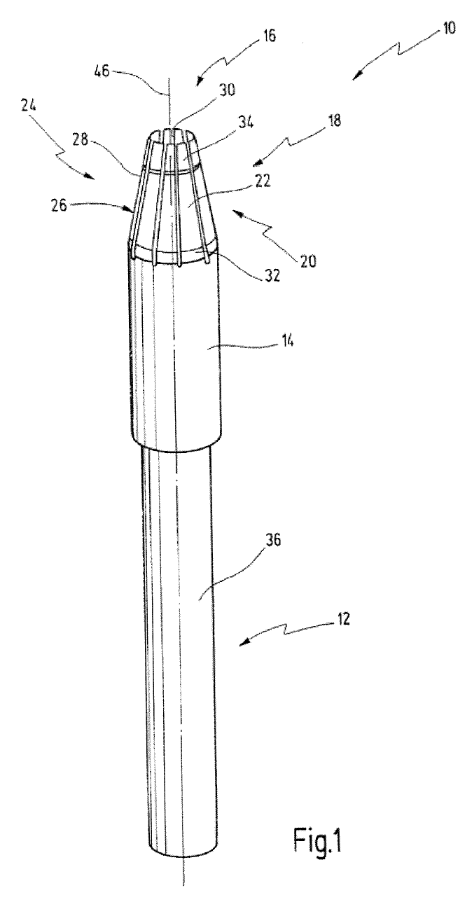

The medical instrument 10 shown in FIG. 1 comprises a cannula 12.

The cannula 12 is received in a sleeve 14 that has a length measuring approximately over a third of the total length of the instrument 10.

At a distal end 16, the sleeve 14 has ten elements 18 which are distributed uniformly about its circumference and which are designed as spreadable elements 20. Depending on the design, it is possible to provide a greater or smaller number of spreadable elements 20, for example at least three or four or six elements.

The spreadable elements 20 are designed as blades 22 that are formed between axially extending slits in the sleeve 14.

The blades 22 are folded together to form a distally tapering body 24, which is designed as a conical body 26.

In the illustrative embodiment shown, the conical body 26 is designed as a truncated cone 28.

At the distal end 16, the truncated cone 28 has an opening 30 through which precise positioning can be achieved.

Each blade 22 is connected to the sleeve 14 via a film hinge 32. The film hinge 32 is created, for example, by making an incision into the material of the sleeve 14.

The blades 22 are pivoted about the film hinge 32.

The blades 22 form a cage in the shape of truncated cone 28.

The material of the sleeve can be a metal or plastic. The sleeve can be designed as a disposable part.

At their distal end, the blades 22 have an outer barb 34, and these serve to create a defined site of force introduction when the blades 22 are spread open.

The medical instrument 10 comprises a spreader 36 designed as a cannula 12 which is received displaceably in the sleeve 14.

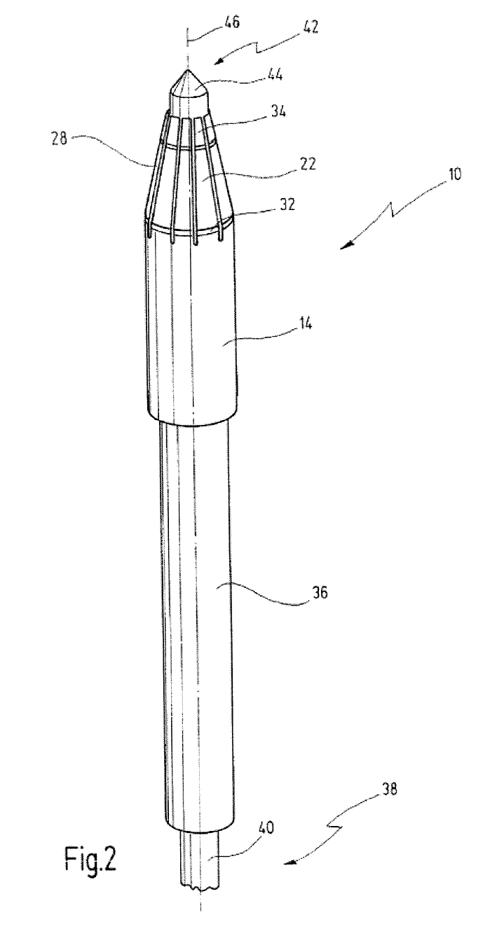

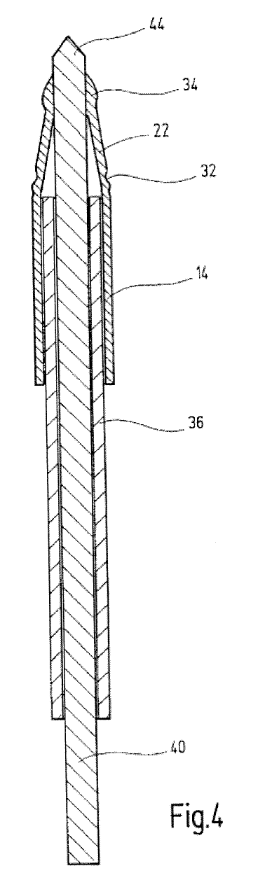

FIGS. 2 and 4 show how a penetrating element 38 is guided through the cannula 12 and through the truncated cone 28.

A penetrating element 38 is designed as a rod 40.

The penetrating element 38 tapers in the direction of its distal end 42 and forms a point 44.

The tip of truncated cone 28 bears closely on the outer side of rod 40.

This construction of the medical instrument 10 simplifies the targeting of the site of application of the instrument 10 and the insertion of the truncated cone 28 between two adjacent vertebrae.

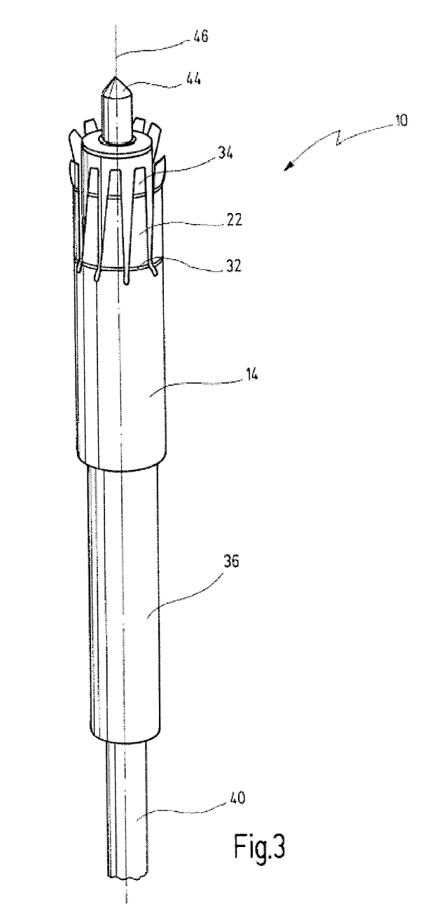

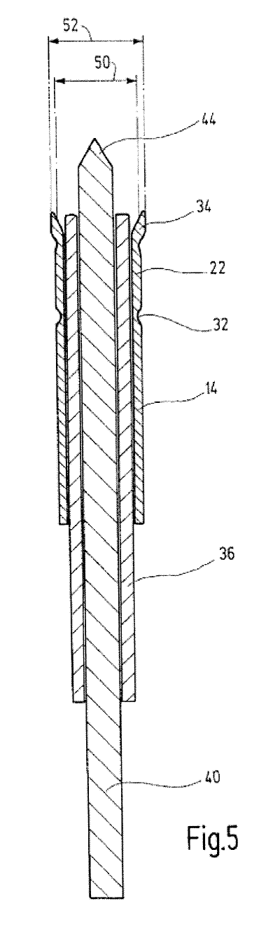

In FIG. 3 and FIG. 5, the medical instrument 10 is shown after the spreadable elements 20 have been spread open.

The spreader 36 designed as a cannula 12 is driven into the tapering body 24 by means of a linear movement along a longitudinal axis 46. This causes the spreadable elements 20 to spread open.

The spreader 36 constitutes a support and reinforcement for the spread-open blades 22 which are subjected to the restoring force of the spread-open vertebrae.

The view in FIG. 5 illustrates how, when the blades 22 are spread open, the outer faces of the barbs 34 form a ring whose external diameter 52 is greater than the external diameter of the sleeve 14. The barbs 34 hook into the bones of the vertebrae and form defined sites of force introduction.

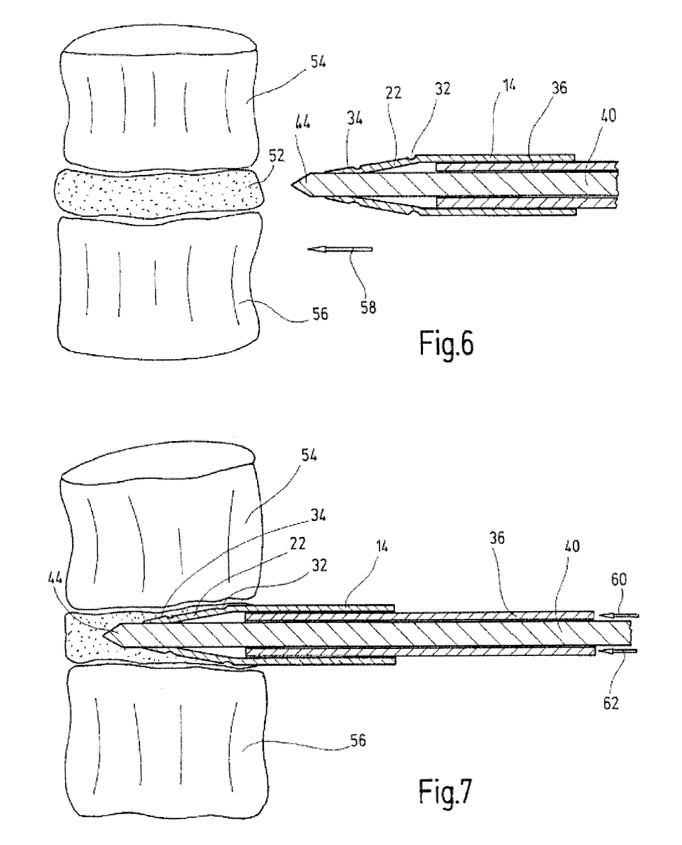

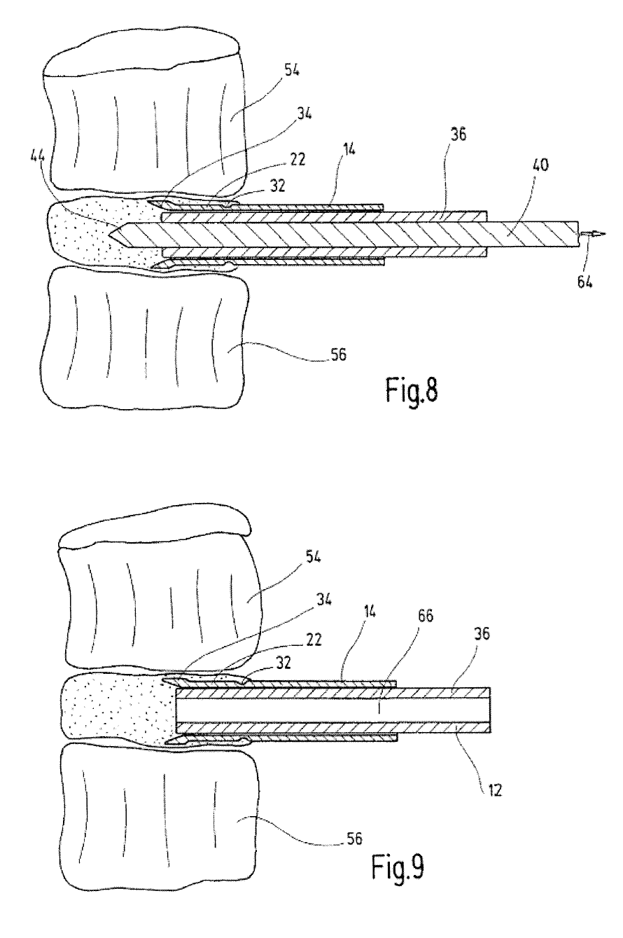

A use of the medical instrument 10 will be briefly explained with reference to FIGS. 6 to 9.

An intervertebral disk 52 is shown in FIG. 6. This intervertebral disk 52 is located between two adjacent vertebrae 54 and 56 of a spinal column.

In the case of an examination or surgical intervention, the medical instrument 10 is now used as follows:

The point 44 of the penetrating element 38 is carefully placed between two adjacent vertebrae 54 and 56. The direction of insertion is indicated by an arrow 58.

The whole unit is then inserted between the vertebrae 54 and 56 by about the entire length of the truncated cone 28.

Then, as is indicated by arrows 60, 62 in FIG. 7, the spreader 36 is moved linearly in the direction of the distal end 16.

The spreader 36 is driven into the truncated cone 28, by which means the spreadable elements 20 are spread open and the adjacent vertebrae 54 and 56 are pushed away from one another and thus kept spaced apart. The spreading movement takes place uniformly and in a defined manner about the film hinges 32.

This is illustrated in FIG. 8.

Thereafter, the penetrating element 38 is withdrawn proximally, as is indicated by an arrow 64.

A channel 66 of the cannula 12 is now free for further maneuvers.

Medical instruments can now be pushed through the channel 66 from proximal to distal, in order to conclude an examination or an intervention.

After an examination or an intervention has been performed, the cannula 12 is drawn back until the blades 22 are bent radially inward again, and the whole unit is then withdrawn.

What is claimed is:

1. A medical instrument for spreading vertebral bodies apart and for operations in an area of a spinal column, comprising
a sleeve,
several spreadable elements protrude from a distal end of said sleeve,
said several spreadable elements are folded together to form a conical body, said conical body is to be inserted between adjacent vertebral bodies of a spinal column, and
a spreader which can be driven into said conical body thereby spreading said several spreading elements, said spreaded elements pushing away said two adjacent vertebral bodies.

2. The medical instrument of claim 1, wherein said conical body is designed as a truncated cone.

3. The medical instrument of claim 1, wherein said spreader is designed as a cannula that is received displaceably within said sleeve.

4. The medical instrument of claim 1, wherein a penetrating element is provided that can be guided through said sleeve and through that conical body.

5. The medical instrument of claim 4, wherein said penetrating element is designed as a rod.

6. The medical instrument of claim 5, wherein said rod having a distal end designed as a point.

7. The medical instrument of claim 1, wherein a penetrating element is provided that can be guided through a cannula received within said sleeve.

8. The medical instrument of claim 7, wherein said penetrating element tapers in a direction of its distal end.

9. The medical instrument of claim 8, wherein said tapering element is designed as a rod having a point.

10. The medical instrument of claim 1, wherein said several spreadable elements are designed as blades.

11. The medical instrument of claim 10, wherein said blades are formed between circumferentially distributed longitudinal slits within said sleeve.

12. The medical instrument of claim 1, wherein each spreadable element is connected to said sleeve via a hinge.

13. The medical instrument of claim 1, wherein said spreadable elements are bent radially inward.

14. The medical instrument of claim 1, wherein each of the spreadable elements has, at its distal end, an outer barb.

15. The medical instrument of claim 14, wherein, when said elements are spread, outer faces of said barbs form a ring, whose external diameter is greater than a diameter of said sleeve.

16. The medical instrument of claim 1, wherein at least three spreadable elements protrude from the distal end of said sleeve.

17. The medical instrument of claim 1, wherein at least four spreadable elements protrude from that distal end of that sleeve.

18. The medical instrument of claim 1, wherein at least six spreadable elements protrude from said distal end of said sleeve.

19. The medical instrument of claim 1, wherein ten spreadable elements protrude from said distal end of said sleeve.
